(19) United States
(12) Patent Application Publication
(10) Pub. No.: US 2026/0334992 A1
(43) Pub. Date: Oct. 29, 2026

(54) TIE SYSTEMS FOR STERNAL CLOSURE

(71) Applicant: REGENTS OF THE UNIVERSITY OF MICHIGAN, Ann Arbor, MI (US)

(72) Inventors: Gardner Yost, Ann Arbor, MI (US); Jeffrey Stephen Plott, Algonac, MI (US); Jonathan Haft, Ann Arbor, MI (US)

(21) Appl. No.: 19/123,354

(22) PCT Filed: Nov. 2, 2023
(86) PCT No.: PCT/US23/36698

§ 371 Date: Apr. 22, 2025

Related U.S. Application Data
(60) Provisional application No. 63/422,729, filed on Nov. 4, 2022.

(51) Int. Cl. A61B 17/82; A61B 17/04

(52) U.S. Cl. CPC A61B 17/823; A61B 17/0401; A61B 2017/0404

(57) ABSTRACT

In accordance with an implementation, a tie assembly includes a needle, a tie, a plug, and a tether. The needle has a proximal end and a distal end and the tie is coupled to the proximal end of the needle. The plug has an axial bore therethrough. The tie is disposed within the bore. The tether tethers the plug to the proximal end of the needle.

### RELATED APPLICATION

This application claims the benefit of and priority to U.S. Provisional Patent Application Number 63/422,729, filed Nov. 4, 2022, the content of which is incorporated by reference herein in its entireties and for all purposes.

### FIELD OF THE DISCLOSURE

The present disclosure relates generally to improved medical devices and techniques for performing tie cerclage closure of the sternum.

### BACKGROUND

Wire cerclage closure is, by far, the most common means for sternal re-approximation. Despite this, sternal wires can cause bleeding from needle holes and can exert excess force on the sternal bone over time, resulting in non-union or dehiscence. These complications can necessitate prolonged operative times, re-operation, blood product administration, and increased levels of care.

Wire cerclage is a simple, inexpensive, and practical means of sternal re-approximation, and, importantly, permits expeditious re-entry into the mediastinum in the case when emergent exploration is required. Several studies utilizing large multicenter cohorts of patients undergoing cardiac surgery with sternotomy have demonstrated that reoperation for excessive bleeding occurs in 5-9% of cases. Poorly controlled sternal bleeding is a common culprit in excessive post-operative bleeding. This may be due to oozing from the sternal incision or the opening of an artery or vein after sternal closure, but often is a result of passage of sternal wires through the bony and soft tissues of the chest. The holes created by the needles and sternal wires are large and are prone to bleeding, as the sternum is a well vascularized area. Some surgeons will attempt to address this bleeding by applying hemostatic agents into the needle holes to attempt to achieve both local tamponade and coagulation. Another approach for ongoing sternal wire bleeding is placement of a compressive suture around each needle hole. This approach is laborious and time-consuming. In addition to the problem of bleeding from needle holes, the use of sternal wires can lead to sternal malunion or dehiscence. This is most often a result of compression of the manubrium or sternal edges by taut stainless-steel wires which erode through the bone over time, leading to loosening and separation of the sternal edges. Sternal malunion is a disfiguring complication, results in significant discomfort, and can compromise respiration. Dehiscence, often complicated by infection, requires surgical intervention in an attempt to reapproximate the sternum. This is a highly morbid complication, and yet the most common solution is reuse of sternal wires to create a woven wire matrix surrounding the sternum and ribcage.

Competing technologies include the sternal wire as currently utilized. This is considered the gold standard. An important barrier to commercialization is potential hesitance of the surgeon to adopt a new device which requires additional time and effort to use. A newer device for sternal closure is the sternal plate, which utilizes titanium plates screwed onto the superficial surface of the sternum. While sternal plating systems have been shown to reduce rates of sternal malunion, they have several drawbacks including cost, time to implement, and difficulty to rapidly re-enter the chest after the sternal plate is secured. Other closure strategies include braided sternal wires and bands which are tightened in a fashion similar to zip-ties.

The use of hollow anchor mechanisms to facilitate wire closure of severed bone and other tissue has been disclosed. Examples include the teachings of U.S. Pat. Nos. 6,033,429, 6,059,818, and 6,302,899 of Cardiac Assist Technologies, Inc. These patents disclose the use of hollow barbed anchors or grommets in severed bone and the subsequent threading of stainless-steel wire or suture material through the bore of each of the anchors or grommets to serve as a lash to close the severed bone and other tissue. Each of these disclosures contemplates a separate instrument in the form of a placing mechanism employed to deploy the anchors or grommets into the bone or other tissue. At the time of deployment, the anchors or grommets are not in communication with wire or suture material. Such a separate placing mechanism suffers from several drawbacks, such as the risk of one or more of the small untethered anchors or grommets falling loose from the placing mechanism, or prematurely dislodging from the bone or other tissue, and becoming lost inside the patient. Given the sharp barbs provided to enhance securement of the anchors or grommets, and the close proximity to critical vessels and organs, this risk is of significant concern. Additionally, the need for a separate applicator represents an inconvenience to the surgeon, additional expense, sterilization challenge, and introduces new technical difficulty as there is limited space for maneuvering of the instruments in the thoracic cavity.

Another drawback of such disclosed placing mechanisms is that placing mechanisms which contemplate deployment of both posterior and anterior anchors or grommets, the bores of such posterior and anterior anchors/grommets are, once deployed, coaxial with one another, posing a practical challenge of threading a sternal wire through small coaxial openings using a C-shaped (or hook-shaped) needle. Alternatively, if a straight needle is employed to thread the sternal wire through the coaxial openings of the posterior and anterior anchors or grommets, manipulating such a straight needle requires application of large forces, great dexterity and risks causing additional bleeding due to the limited space behind the anterior thoracic wall to accommodate the length of the needle.

Certain of the disclosed anchors or grommets require crimping in order to ensure securement in the bone or other tissue, which detrimentally requires the use of yet an additional crimping instrument, thereby prolonging the duration of the surgery.

U.S. Pat. No. 8,062,295, to McDevitt (DePuy Mitek, Inc.), discloses the use of a hollow cylinder, which the patent refers to as an “eyelet,” that is secured with suture material, interference fit, or surgical epoxy, into a transosseous tunnel, for use in rotator cuff reconstructive surgery.

The manner in which the above drawbacks, and others, in the prior art can be overcome by the constructions and techniques of the present disclosure are detailed below.

### SUMMARY

The present disclosure relates to sternal tie systems incorporating hemostatic, force distributing elements. There is a need for a system that reduces both blood loss from sternal needle holes and rates of sternal dehiscence as a result of bone cutting, as such a system would shorten operative times, improve aggregate patient outcomes, and reduce surgical and post-surgical costs.

The device of the present disclosure utilizes a plug, which is fed into a needle hole in the sternum around a length of sternal tie. By advancing the plug adjacent to the needle hole, there is tamponade of bleeding around that hole, particularly as the tie is tightened. Under normal circumstances there is direct abutment of the tightened tie against the boney surface.

This plug device advantageously distributes the forces applied by the tie onto a larger surface area of bone. This should reduce rates of tie erosion into bone and maintain long-term sternal approximation. Return to the operating room confers additional risk to the patient in the form of anesthesia, mechanical ventilation, re-do sternotomy, and potential re-utilization of cardio-pulmonary bypass. There is also increased need for red blood cell and coagulation factor transfusion as well as other intravenous medications which target hemostasis and blood pressure control. Not surprisingly, the mortality rate in these patients is appreciably higher compared to patients who need no additional surgical intervention. It is assumed that use of this device, by reducing volume of post-closure blood loss and improving long-term stability of the tie reinforced sternum, will result in lower rates of re-operation.

The sternal tie assembly of the present disclosure includes at least one needle, such as a C-shaped or hook-shaped, having a distal end capable of piercing and penetrating hard and semi-hard tissue, including one or more of sternum, manubrium, ribs, rib cartilage, and intercostal muscle and a proximal end from which a length of sternal tie extends. The distal end may be sharp or blunt. Also extending from a proximal end of the needle is suture material, that may be in the form of at least two suture lines each being secured at a first end to the proximal end of the needle and at a second end to a head of a posterior plug. One suture line or more than two suture lines may alternatively be used. The posterior plug is slidably mounted along the sternal tie. In one embodiment, the posterior plug has a plurality of suture-receiving holes through the head, which are occupied by the suture lines to tether the posterior plug to the proximal end of the needle.

In some implementations, the sternal tie assembly is provided with two needles, one on either end of the sternal tie, each of the needles having a respective posterior plug slidably received on the sternal tie and tethered to the proximal end of that needle by suture material.

The tether may be attached to a portion of the sternal tie that is near the proximal end of the needle but not directly attached to the proximal end of the needle, for example.

In use, the assembly is removed from a sterile package, and a distal end of each of the needles is inserted through respective exposed sternum, manubrium, rib, rib cartilage, intercostal muscle, or other tissue that has been separated for an invasive surgical procedure, such as a sternotomy. The needles may be inserted from a posterior side of the tissue toward an anterior side, but insertion of one or both of the needles is within the scope of the present disclosure. After each needle is advanced through the respective tissue, forming a hole through the tissue, the hole is immediately occupied by the sternal tie and suture material extending from the proximal end of the needle. Inasmuch as both needle sides are manipulated in the same manner, for ease of description, only one needle side is discussed in the following steps, but it is to be understood that the same operations are to be performed on both sides of the separated sternum, manubrium, rib, rib cartilage, intercostal muscle, or other tissue. The needle, tie, and suture material are continually advanced past the tissue until the respective posterior plug is nearly drawn into the hole through the tissue.

Next, the needle is severed from the sternal tie and suture material. After removal of the needle, the suture material is pulled to further advance the posterior plug toward the hole formed by the needle. Next, an anterior plug having a bore that extends through an open head of the anterior plug is threaded along the exposed sternal tie and advanced in a posterior direction until the anterior plug is aligned with, and in close proximity to, an anterior opening of the hole through the tissue. The head of the anterior plug may be provided with one or more grooves or cleats in a perimeter thereof, to facilitate securement and tightening of the suture material. The suture material is then used to draw the anterior plug adjacent to the anterior opening of the hole and abutting the tissue, and is continually tightened until both the posterior and anterior plugs are snugly secured relative to respective sides of the hole through the tissue. The suture material is secured to the head of the anterior post, such as by tying an appropriate knot or set of knots that can reliably maintain position of the plugs relative to the hole in the tissue.

The sternal tie is bent in the direction of the other end of the sternal tie extending from the other side of the tissue to be closed, and once the two ends of the tie are brought together, the tie ends are twisted or otherwise coupled, thereby bringing the two ends of the tissue toward one another, until closure is achieved. The posterior and anterior plugs secured relative to the tissue serve to buffer the tissue against the sternal tie causing bone cutting, which can detrimentally lead to sternal malunion and bleeding.

In accordance with a first implementation, a tie assembly includes a needle, a tie, a plug, and a tether. The needle has a proximal end and a distal end and the tie is coupled to the proximal end of the needle. The plug has an axial bore therethrough. The tie is disposed within the bore. The tether tethers the plug to the proximal end of the needle.

In accordance with a second implementation, a tie assembly includes a needle, a tie coupled to the needle, a plug having an axial bore therethrough, and a tether tethering the plug to the needle or the tie. The tie disposed within the bore.

In further accordance with the first and/or second implementations, an apparatus and/or method may further include or comprise any one or more of the following:

In accordance with an implementation, the tie includes a wire.

In accordance with another implementation, the needle, the tie, and the tether are crimped together.

In accordance with another implementation, the tie assembly also includes a second needle, a second plug, and a second tether. The second needle has a proximal end and a distal end and the tie extends from the proximal end of the second needle. The second plug has an axial bore therethrough. The tie is disposed within the bore and the second tether tethers the second plug to the proximal end of the needle.

In accordance with another implementation, the needle and the second needle are hook-shaped.

In accordance with another implementation, the plug includes a pair of holes and the tether includes a first end, a central portion, and a second end. The first end and the second end of the tether are coupled to the proximal end of the needle and the central portion of the tether passes through the pair of holes.

In accordance with another implementation, the plug defines chamfers or fillets at openings of the holes.

In accordance with another implementation, the plug has a top surface that defines an opening of the bore, a bottom surface, and a curved surface that defines the bore and curves outwardly from the opening of the bore at the top surface toward the bottom surface.

In accordance with another implementation, the top surface is convex.

In accordance with another implementation, the bottom surface is convex.

In accordance with another implementation, the bottom surface faces the needle.

In accordance with another implementation, the needle is removable from the tie and the tether, and the tie assembly also includes a second plug having an axial bore and threadable onto an exposed end of the tie upon removal of the needle.

In accordance with another implementation, the second plug has a top surface that defines an opening of the bore, a bottom surface, and a curved surface that defines the bore and curves outwardly from the opening of the bore at the top surface toward the bottom surface.

In accordance with another implementation, the second plug includes a pair of grooves and the tether has portions that pass through the pair of grooves.

In accordance with another implementation, the second plug includes a perimeter and the grooves include tapered surfaces, an entrance, and an opening. The tapered surfaces extend inward from the perimeter of the second plug toward the entrance of the opening of the corresponding groove.

In accordance with another implementation, the second plug includes a pair of protrusions extending from the bottom surface and positioned on opposite sides of the bore.

In accordance with another implementation, the protrusions have a triangular cross-section.

In accordance with another implementation, the protrusions define a groove between the protrusions.

In accordance with another implementation, the protrusions include outwardly tapered walls.

In accordance with another implementation, the tie interacts with the outwardly tapered walls to urge the tie to be positioned within the groove.

In accordance with another implementation, the grooves each include a first portion and a second portion that are off-set from one another.

In accordance with another implementation, the first portion and the second portion are radially spaced from one another.

In accordance with another implementation, the apparatus includes a second needle from which the tie extends, a plate, and a pair of second plugs each having an axial bore and threadable onto exposed ends of the tie upon removal of the needles.

In accordance with another implementation, the plate is to extend across opposing ends of tissue.

In accordance with another implementation, the plate is positionable between the second plugs and the tissue.

In accordance with another implementation, the second plugs are positionable between plate and the tissue.

In accordance with another implementation, the second plugs nest within the plate.

In accordance with another implementation, the plate has opposing end slots through which the tie passes.

In accordance with another implementation, the second plugs are linearly movable along the end slots to accommodate different sternal widths.

In accordance with another implementation, the plate and the second plugs include corresponding teeth that mate with each other and enable linear adjustment of the second plugs along the plate.

In accordance with another implementation, the apparatus includes a second plug plate assembly.

In accordance with another implementation, the second plug plate assembly has a pair of plates each including a second plug.

In accordance with another implementation, the plates are coupled.

In accordance with another implementation, the plates are coupled by a tongue-and-groove connection.

In accordance with another implementation, the tether is tethered to the proximal end of the needle directly.

In accordance with another implementation, the tether is tethered to the proximal end of the needle indirectly.

### BRIEF DESCRIPTION OF THE SEVERAL VIEWS OF THE DRAWING

FIG. 1 is a perspective view of a wire closure following a sternotomy using a plurality of conventional (prior art) sternal wires.

FIG. 2 is a plan view of a conventional needle having a sternal wire integral with, and extending from, a proximal end thereof.

FIG. 3 is a plan view of a tie assembly that includes a needle, a tie, a plug, and a tether in accordance with the teachings of this disclosure.

FIG. 4 shows a detailed view of the needle, the tie, and the tether crimped together at the proximal end of the needle.

FIG. 5 shows a detailed view of the tie extending through the plug and the second plug and the tether and the second tether coupled to the corresponding plugs.

FIG. 6 is a cross-sectional view of the plug of FIG. 3.

FIG. 7 shows the tie assembly of FIG. 3 with the needle removed and a second plug having an axial bore that is threaded onto an exposed end of the tie upon removal of the needle, securement of the anterior plug, and twisting of the free ends of the wire.

FIG. 8 shows a bottom isometric view of the second plug of FIG. 3.

FIG. 9 shows a bottom isometric view of the plug of FIG. 3.

FIGS. 10-17 illustrate a method of use of the tie assembly of the present disclosure.

FIGS. 18-20 illustrate different views of the second plug of FIG. 3.

FIG. 21 illustrates an isometric top view of the plug of FIG. 3.

FIG. 22 illustrates a cross-sectional view of the second plug of FIG. 3.

FIG. 23 illustrates a bottom isometric view of the second plug.

FIG. 24 illustrates a bottom isometric view of the plug.

FIGS. 25A and 25B show anterior and posterior views of a sternal model using a plurality of the tie assemblies and plugs as disclosed herein.

FIG. 26 is an isometric view of a second plug that can be used to implement the tie assembly of FIG. 3.

FIG. 27 is an isometric view of a plate and a pair of the second plugs that can be used to implement the tie assembly of FIG. 3.

FIG. 28 is an isometric view of the plate and the plugs of FIG. 27 showing the second plugs positioned between the plate and the tissue.

FIG. 29 is an isometric view of another plate and a pair of second plugs that can be used to implement the tie assembly of FIG. 3.

FIG. 30 shows a cross-sectional view of a tie assembly including the plate and the second plugs of FIG. 29.

FIG. 31 is an isometric view of a second plug plate assembly that can be used with the tie assembly of FIG. 3.

FIG. 32 is a bottom view of the plate of the second plug plate assembly of FIG. 31 showing the second plug.

### DETAILED DESCRIPTION

The present disclosure relates generally to improved medical devices and techniques for performing tie cerclage closure of the sternum, and more particularly, to a sternal tie assembly having at least one integral needle, a sternal tie extending from a proximal end of the needle, suture material secured to the needle, and a plug tethered to the needle, such that after passage of the needle through sternum, manubrium, ribs, costal cartilage, intercostal muscle, or other tissue, the needle and suture material can be pulled to advance the posterior plug into or adjacent to a posterior entry of the formed needle hole. The suture material may be directly or indirectly secured to the needle, for example. Further aspects of the disclosure include a second, anterior plug that can be advanced adjacent to an anterior exit of the formed needle hole. The second, anterior plug may be provided with a plug head with one or more cleats or grooves in a perimeter thereof to facilitate tightening of the suture material so as to draw the posterior and anterior plugs toward one another and tightly secure them in place adjacent to the needle hole through the sternum, manubrium, rib, costal cartilage, intercostal muscle, or other tissue.

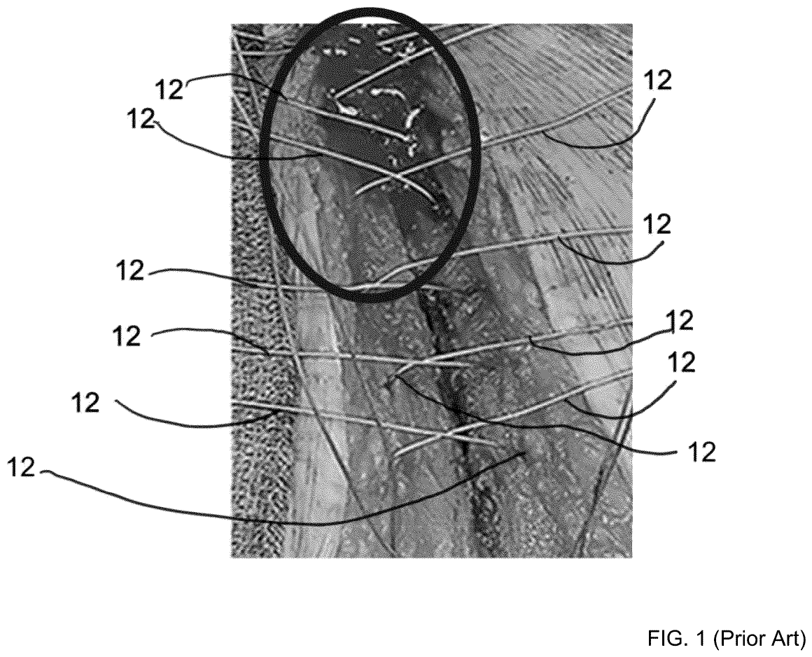

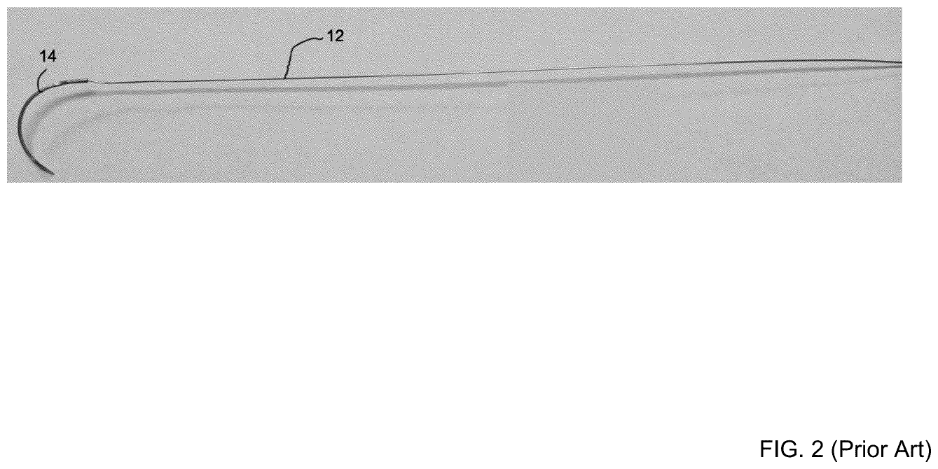

With reference to FIG. 1, a wire cerclage closure using a conventional set of sternal wires is illustrated. In a typical procedure, a set of approximately seven J-shaped (i.e., hook-shaped) needles, each integral with a respective sternal wire 12 extending from a proximal end thereof (illustrated in FIG. 2), is inserted through sternum, manubrium, rib, rib cartilage, intercostal muscle, or other tissue. Each of the sternal wires 12 is then crossed with a sternal wire 12 of a needle inserted through the sternum, manubrium, rib, rib cartilage, intercostal muscle, or other tissue on the other side of the sternal separation, then the sternal wires 12 are twisted until closure is achieved. While efforts have been made to develop grommets, cylinders, or other inserts to provide such isolation, as discussed in the BACKGROUND section of this disclosure, these efforts have heretofore not provided a satisfactory solution to the problems of delivery of such devices to the holes in the tissue formed by the needle, and securement of the devices to the tissue.

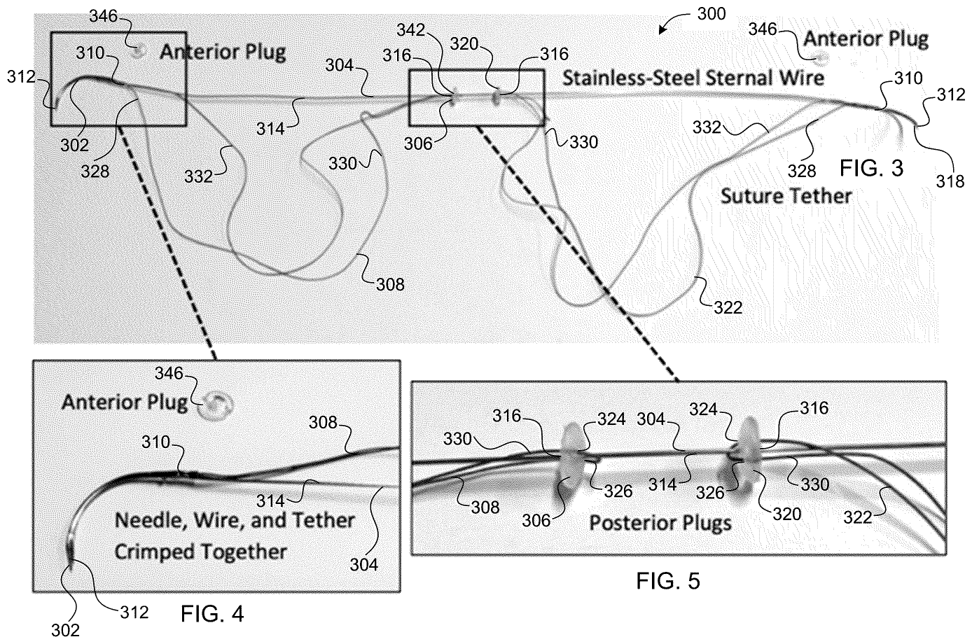

FIG. 3 is a plan view of a tie assembly 300 that includes a needle 302, a tie 304, a plug 306, and a tether 308 in accordance with the teachings of this disclosure. The needle 302 has a proximal end 310 and a distal end 312. The tie 304 is coupled to and/or extends from the proximal end 310 of the needle 302 in the implementation shown. The tether 308 may or may not be directly connected to the proximal end 310 of the needle 302. For example, the tether 308 may be connected to the tie 304 but not directly the needle 302 in some implementations. The tether 308 may be bonded, welded, and/or otherwise secured to the tie 304 near the proximal end 310 of the needle 302 in this implementation. The tether 308 may be directly connected to the needle 302 as shown. The tie 304 may be referred to as a sternal tie and may be implemented by a wire 314. The wire 314 may be a stainless-steel sternal wire as an example. The tie 304 may alternatively be a string, a cord, a braid, or anything used for fastening and may include, for example, stainless steel, another metal, and/or plastic, such as example, polyetheretherketone (PEEK), polyethylene, or polyester. The tie 304 may have any cross-section including a circular cross-section, an oblong cross-section, and/or a rectangular cross-section.

The plug 306 has an axial bore 316 therethrough and the tie 304 is disposed within the bore 316. The plug 306 may be referred to as a posterior plug. The tether 308 tethers the plug 306 to the proximal end 310 of the needle 302. The tether 308 may be referred to as suture material. The needle 302, the tie 304, and the tether 308 are crimped together in the implementation shown. The needle 302, the tie 304, and the tether 308 may be coupled together in other ways, however.

The tie assembly 300 also includes a second needle 318 having a proximal end 310 and a distal end 312. The tie 304 is coupled to and/or extends from the proximal end 310 of the second needle 318. The tie assembly 300 also includes a second plug 320 having an axial bore 316 therethrough. The second plug 320 may be referred to as a posterior plug. The tie 304 is disposed within the bore 316 and a second tether 322 tethers the second plug 320 to the proximal end 310 of the needle 318. The needle 302 and the second needle 318 are hook-shaped in the implementation shown. The needle 302 and/or the second needle 318 may be implemented by a different type of needle, however.

FIG. 4 shows a detailed view of the needle 302, the tie 304, and the tether 308 crimped together at the proximal end 310 of the needle 302.

FIG. 5 shows a detailed view of the tie 304 extending through the plug 306 and the second plug 320 and the tether 308 and the second tether 322 coupled to the corresponding plugs 306, 320. Each of the plugs 306, 320 has a pair of holes 324, 326 and the tether 308 has a first end 328 (See, FIG. 3), a central portion 330, and a second end 332 (see, FIG. 3). The first end 328 and the second end 332 of the tether 308 are coupled to the proximal end 310 of the needle 302 and the central portion 330 of the tether 308 passes through the pair of holes 324, 326. The coupling between the tethers 308, 322 and the plugs 306, 320 via the holes 324, 326 allows the plugs 306, 320 to be moved along the tie 304 toward the corresponding needle 302, 318 in response to a healthcare worker (e.g., surgeon) pulling on the tethers 308, 322 to position the plugs 306, 320 in place against a posterior side of the tissue being fastened together.

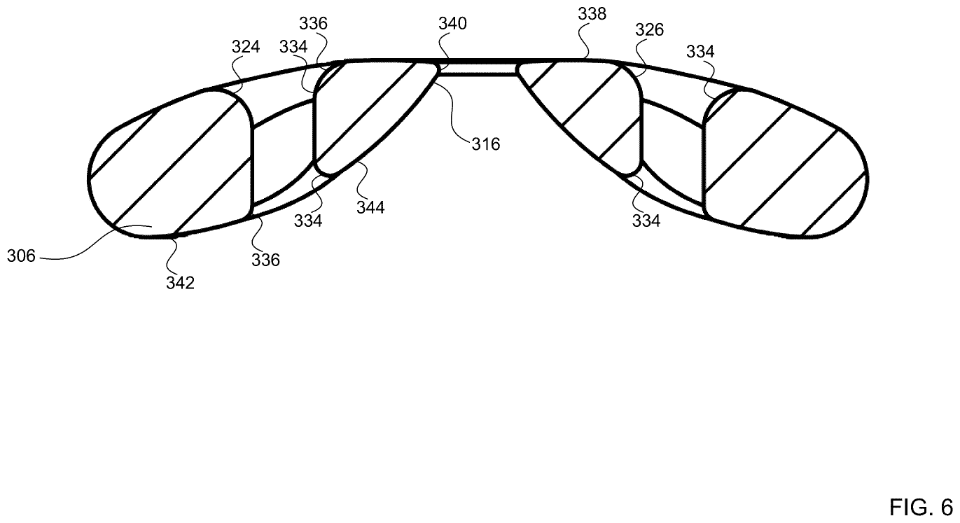

FIG. 6 is a cross-sectional view of the plug 306 of FIG. 3. The plug 306 includes the bore 316 and the holes 324, 326 and the plug 306 defines fillets 334 at openings 336 of the holes 324, 326 in the implementation shown. The fillet may be a chamfer and/or a contoured surface as examples. The plug 306 also has a top surface 338 that defines an opening 340 of the bore 316, a bottom surface 342, and a curved surface 344 that defines the bore 316 and curves outwardly from the opening 340 of the bore 316 at the top surface 338 toward the bottom surface 342. The top surface 338 is convex and the bottom surface 342 is convex and faces the needle 302 of the of the tie assembly 300 as shown in FIG. 3. The surfaces 338, 342 may be different than convex and/or concave and the plug 306 may be differently oriented relative to the needle 302. The bottom surface 342 may form a pocket in which the tissue 349 may be received.

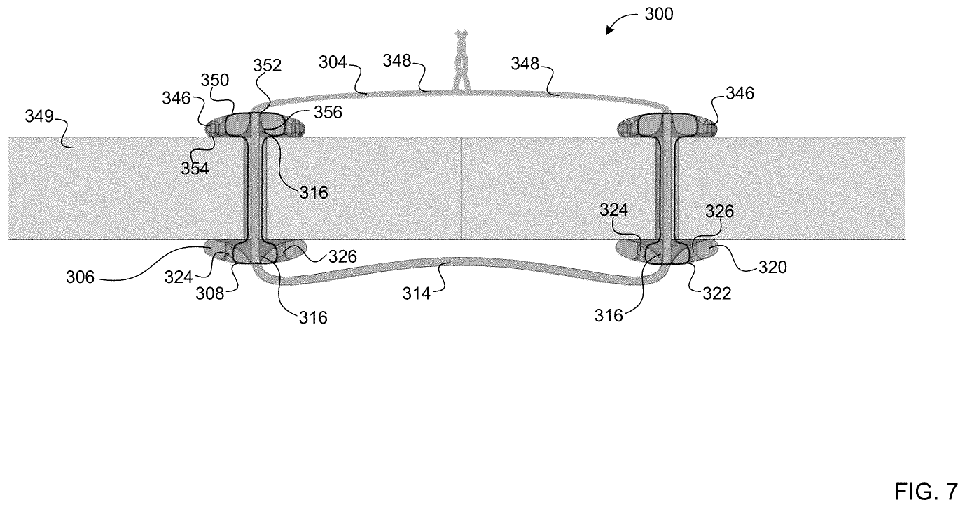

FIG. 7 shows the tie assembly 300 of FIG. 3 with the needle 302 removed and a second plug 346 having an axial bore 316 threaded onto an exposed end 348 of the tie 304.

The second plug 346 may be referred to as an anterior plug. The tie assembly 300 and the plugs 346 are used to secure opposing ends of tissue 349 together.

The second plug 346 has a top surface 350 that defines an opening 352 of the bore 316, a bottom surface 354, and a curved surface 356 that defines the bore 316 and curves outwardly from the opening 352 of the bore 316 at the top surface 350 toward the bottom surface 354.

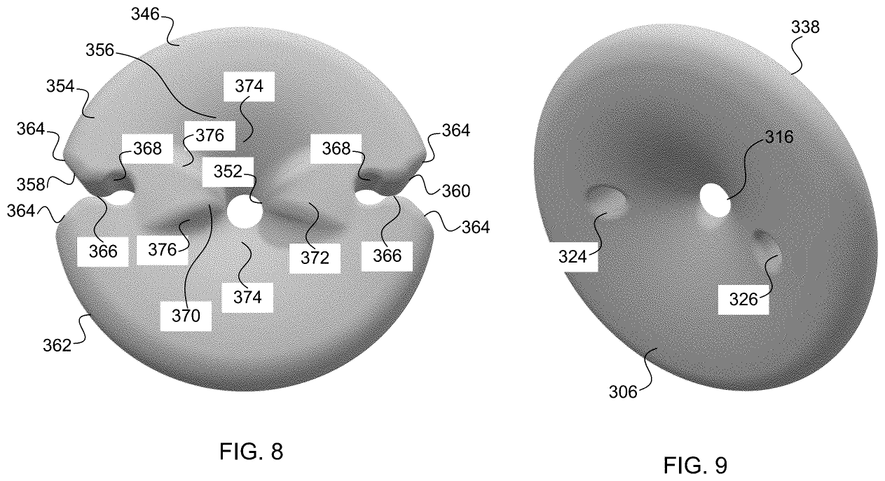

FIG. 8 shows a bottom isometric view of the second plug 346 of FIG. 3. The second plug 346 has a pair of grooves 358, 360. The tether 308 has portions that pass through the pair of grooves 358, 360 when securing the opposing ends of the tissue 349 together. The grooves 358, 360 may be referred to as cleats. The second plug 346 includes a perimeter 362 and the grooves 358, 360 include tapered surfaces 364, an entrance 366, and an opening 368. The tapered surfaces 364 extend inward from the perimeter 362 of the second plug 346 toward the entrance 366 of the opening 368 of the corresponding groove 358, 360. The grooves 358, 360 may be differently structured than shown, however.

The second plug 346 also includes a pair of protrusions 370, 372 extending from the bottom surface 354 and positioned on opposite sides of the bore 316 in the implementation shown. The protrusions 370, 372 may have a triangular cross-section as shown or another cross section.

The protrusions 370, 372 define a groove 374 between the protrusions 370, 372 and have outwardly tapered walls 376. The protrusions 370, 372 may increase the structural integrity of the second plug 346 and allow the axial bore 316 to have a relatively small height.

The axial bore 316 having a reduced height allows the second plug 346 to relatively easily move on the tie 304 even if a kink is present on the tie 304 as an example. The tie 304 interacts with the outwardly tapered walls 376 in practice to urge the tie 304 to be positioned within the groove 374. The tie 304 being positioned within the groove 374 applies the corresponding force onto the areas of the second plug 346 defining the groove 374 and reduces the likelihood of the second plug 346 being damaged in use.

FIG. 9 shows a bottom isometric view of the plug 306 of FIG. 3.

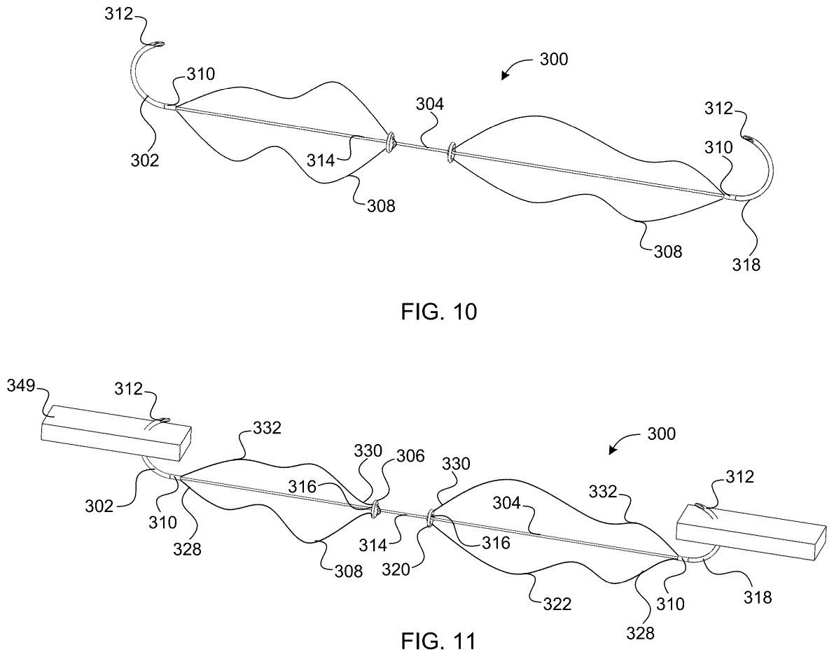

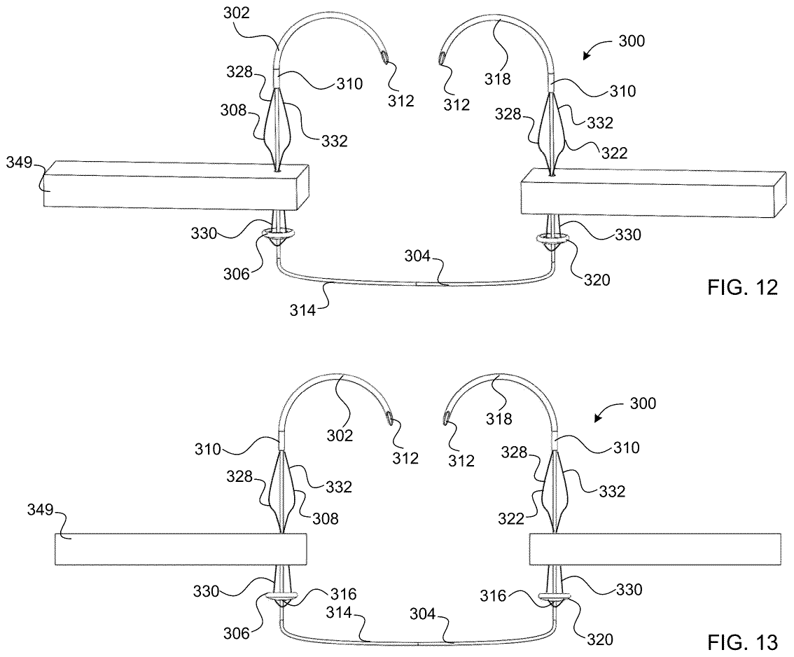

FIGS. 10-17 illustrate a method of use of the tie assembly 300 of the present disclosure that involves first, inserting the distal end 312 of each of the needles 302, 318 of the tie assembly 300 through one of the two exposed ends of the sternum, manubrium, rib, rib cartilage, intercostal muscle, or other tissue 349 that are desired to be closed toward one another. The needles 302, 318 are then advanced through the holes formed through the tissue 349 as illustrated in FIGS. 11-13, and once the needles 302, 318 are pulled out an anterior side of the tissue 349, the trailing tie 304 and the tether 308 that are secured to or integral with the proximal end of the associated needle 302 necessarily follow. Advantageously, continued advancement of the needles 302, 318 and the tether 308 brings the tethered plugs 306, 320 into alignment with the one of the holes through the tissue 349 just formed by the needles 302, 318, which holes are maintained by the tie 304.

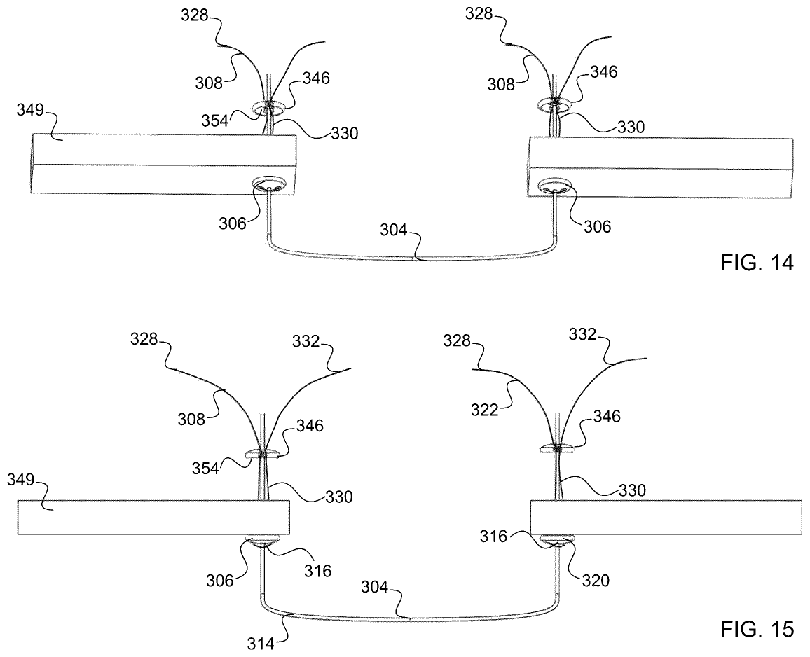

The needles 302, 318 are then severed from the tie 304 and from the tether 308, as illustrated in FIGS. 14 and 15. By pulling the tether 308 anteriorly, the surgeon may continue pulling on the tether 308 until resistance is encountered, likely indicating the plug 306 has made contact with the posterior side of the tissue 349, as illustrated in FIGS. 14 and 15. The surgeon may also confirm that the posterior plug 306 is secure and in place by reaching one or more fingers around the sternal edge and palpating the location and positioning of the plug 306 relative to the tissue 349. The plug 306 may thus seat against an entrance formed by the needle hole but the plug 306 may not enter or substantially enter the needle hole. The interaction between the plug 306 and the needle hole may reduce or inhibit bleeding.

Next, an anterior plug 346 is threaded onto each exposed end 348 of the tie 304, as illustrated in FIGS. 14 and 15, with the bottom surface 354 facing the anterior end of the hole formed through the tissue 349. The anterior plug 346 is also referred to as the second plug 346. To facilitate securement of the tether 308 to the anterior plug 120, instead of holes 324, 326, like in the posterior plug 306, 320, each of the anterior plugs 346 is provided with a pair of radially inwardly-directed grooves 358, 360 (see, FIG. 8). The grooves 358, 360 may be referred to as suture-receiving grooves or tether receiving grooves. The grooves 358, 360 may serve as a continuous cleat or synching mechanism, permitting the tether 308 to tighten, but not loosen, as the plugs 306, 320, 346 are secured relative to and/or within the hole or channel formed through the tissue 349 by the needle 302, 318.

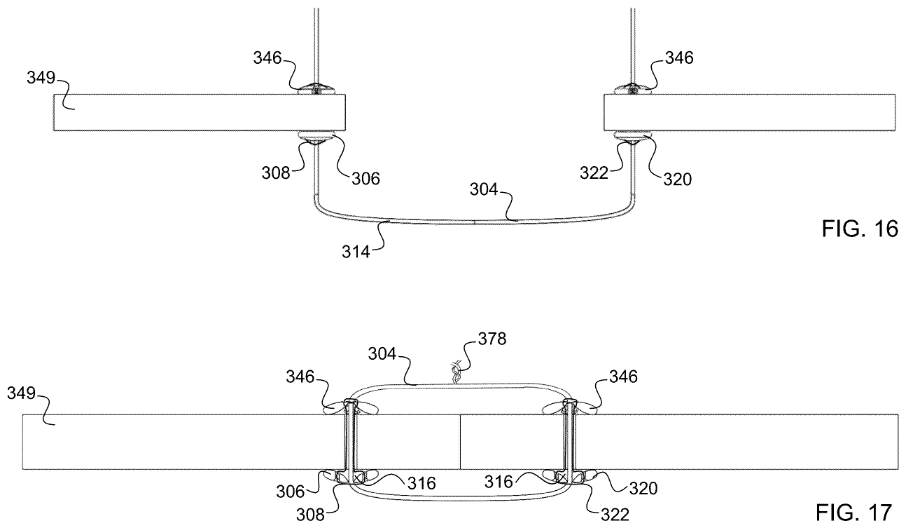

The tether 308 is used to secure the anterior plugs 346 to the tissue 349 as shown in FIG. 16, and then the exposed ends of the tie 304 are brought together and twisted 378 as shown in FIG. 17, thereby promoting closure of the two ends of the tissue 349. In this implementation, the ends of the tether 308 may be tied together, pulling both the posterior and anterior plugs 306, 320, 346 together and substantially ensuring that the plugs 306, 320, 346 do not dislodge or migrate over time.

In securing the anterior plugs 346 to the tissue 349, there are two suture knot options that will work well for this application, both of which are intended to maintain tension in the suture as the knot is tied. 1) A surgeon's knot (this is a reef knot with the application of an extra twist to yield a double overhand knot), followed by 2-3 additional reef knots, tied in alternating directions. Alternatively, 2) A slip knot (this is two reef knots, tied in the same direction so that the knot can be slipped down onto the structure around which the suture is placed), followed by 2-3 additional reef knots, tied in alternating directions.

The entirety of the sternal tie assembly 300 of the present disclosure, or one or more of the tie 304, the tether 308, and the posterior plugs 306, 320 and anterior plugs 346 thereof, may be made of a biocompatible material intended for permanent indwell. Alternatively, one or more of these components of the tie assembly 300 may be made of a bioabsorbable material, dissolving in vivo over time. Alternatively, one or more of these components may be made of a hemostatic material which facilitates and expedites local blood clotting.

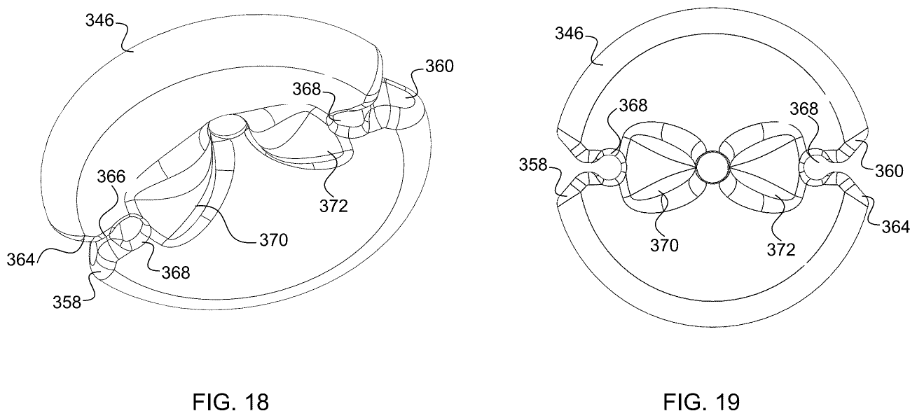

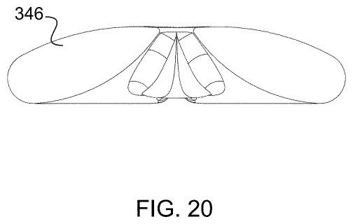

FIGS. 18-20 illustrate different views of the second plug 346 of FIG. 3. FIG. 20 is a cross-sectional view of the second plug 346.

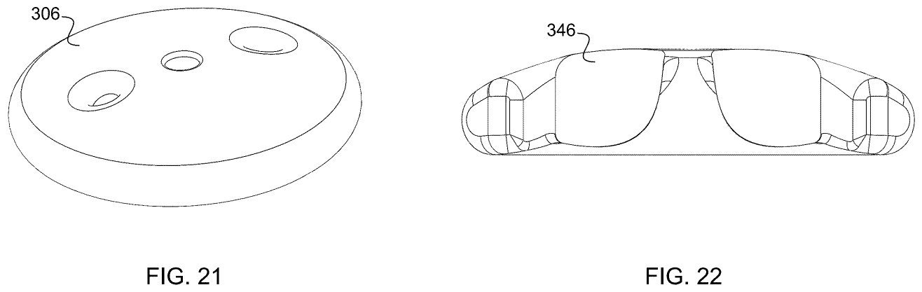

FIG. 21 illustrates an isometric top view of the plug 306 of FIG. 3.

FIG. 22 illustrates a cross-sectional view of the second plug 346 of FIG. 3.

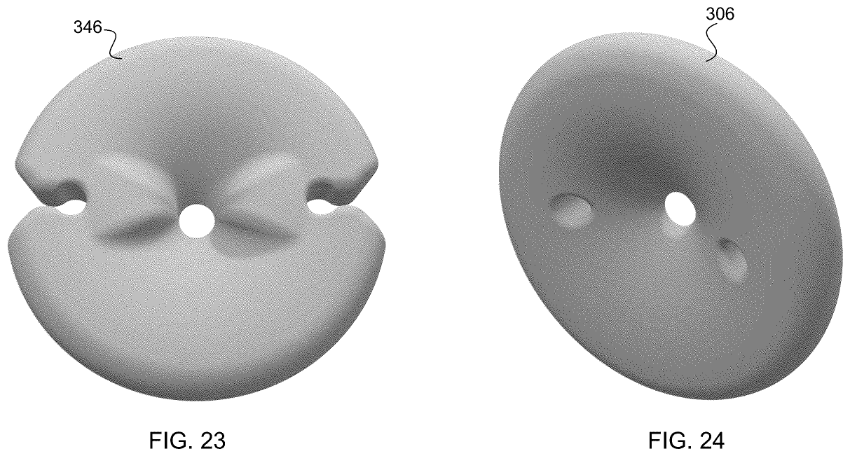

FIG. 23 illustrates a bottom isometric view of the second plug 346.

FIG. 24 illustrates a bottom isometric view of the plug 306.

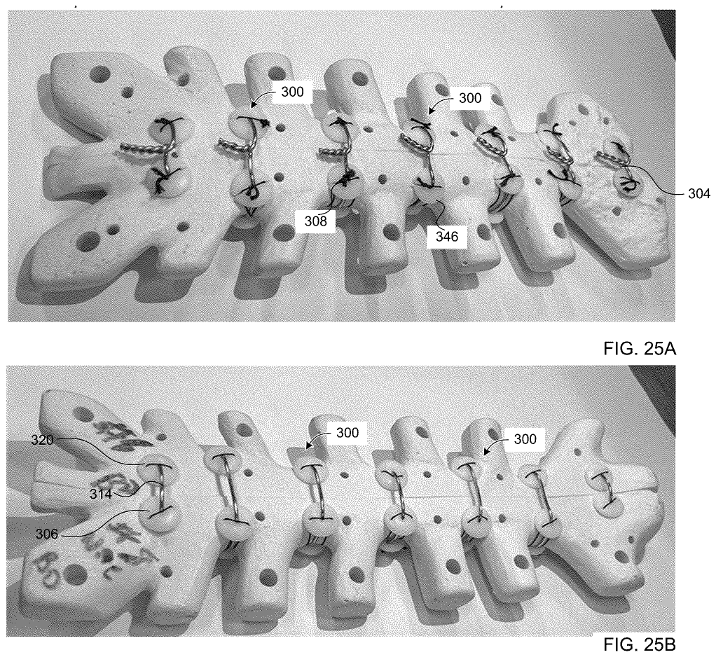

FIGS. 25A and 25B show anterior and posterior views of a sternal model with a plurality of the tie assemblies 300 and plugs 306, 320, 346 as disclosed herein. The ties 304 are oriented approximately 90° relative to the tethers 308 that are positioned through the holes 324, 326 of the second plugs 346 and that are used to secure the second plugs 346 relative to the tissue 349. The ties 304 may interact with the protrusions 370, 372 on the bottom surface 354 of the second plugs 346 to position the ties 304 within the grooves 374 of the second plugs 346 and orient the second plugs 346 such that the ties 304 are oriented approximately 90° relative to the tethers 308.

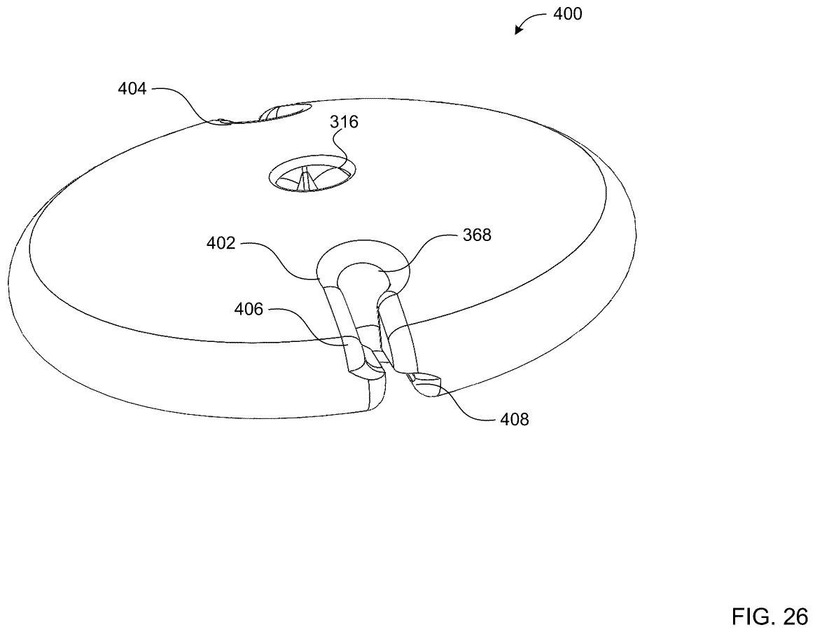

FIG. 26 is an isometric view of a second plug 400 that can be used to implement the tie assembly 300 of FIG. 3. The second plug 400 includes a pair of grooves 402, 404 and the tether 308 has portions that pass through the pair of grooves 402, 404. Each of the grooves 402, 404 has a first portion 406 and a second portion 408 that are off-set from one another. The first portion 406 and the second portion 408 are connected. The portions 406, 408 being off-set forms a cleat that deters the tether 308 from coming out of the grooves 402, 404 and enables the tether 308 to be retained within the opening 368, for example. The first portion 406 and the second portion 408 are radially spaced from one another in the implementation shown.

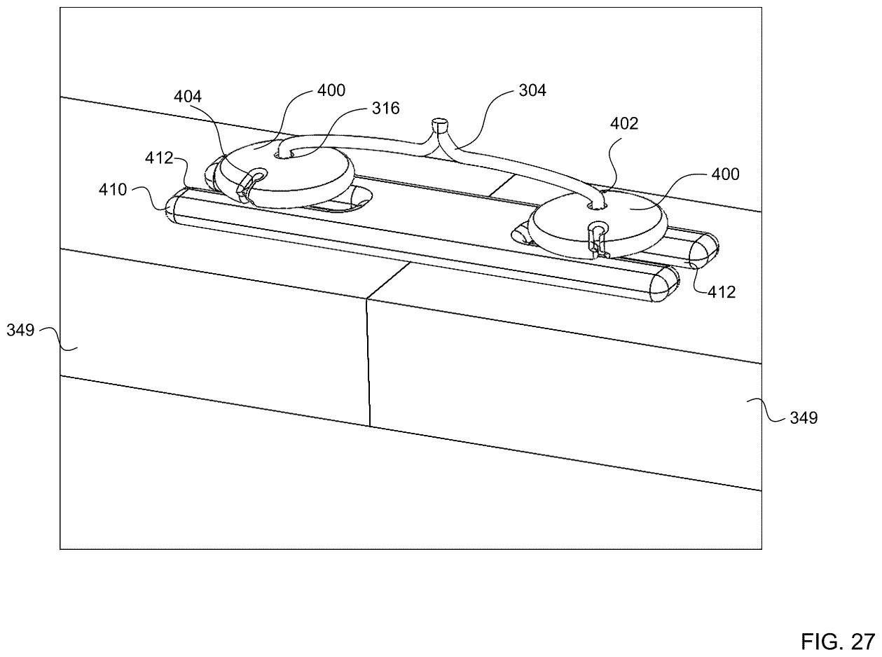

FIG. 27 is an isometric view of a plate 410 and a pair of the second plugs 400 that can be used to implement the tie assembly 300 of FIG. 3. The plate 410 acts as a stiffener and extends across opposing ends of the tissue 349. The plate 410 is positioned between the second plugs 400 and the tissue 349 in the implementation shown. The second plugs 400 may rest on top of the plate 410 and/or nest within the plate 410. The plate 410 has opposing end slots 412 through which the tie 304 passes. The plate 410 including the slots 412 is H-shaped as shown. The plate 410 may have an alternative configuration, however.

The second plugs 400 are linearly movable along and/or relative to the end slots 412 to accommodate different sternal widths. The second plugs 400 may be moved toward or away from one another depending on the sternal width, for example. The plate 410 may be configured so that excess plate elements can be trimmed to length depending on the sternal width of the patient. The plate 410 may include lines of weakness to facilitate excess plate elements being trimmed in some implementations. The plate 410 may be made of a variety of biocompatible materials, such as PEEK and/or metals. The position of the plate 410 in FIG. 27 may be more secure and provide increased strength as an example.

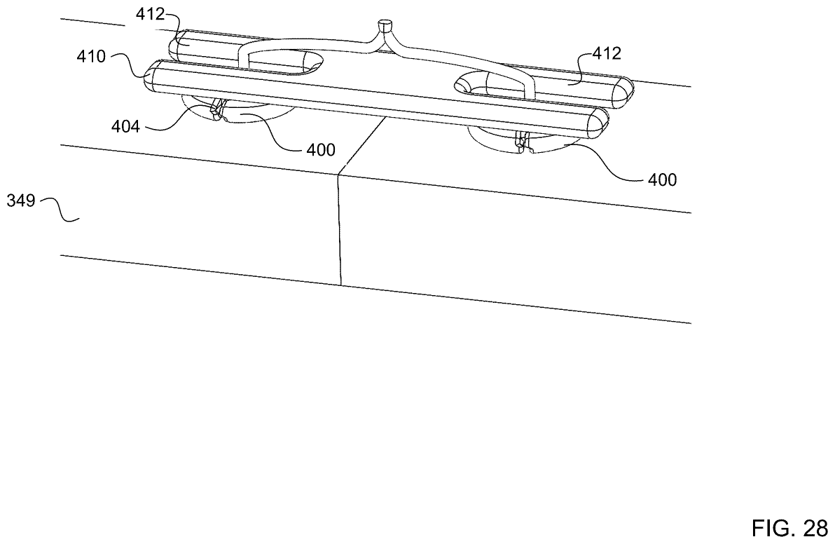

FIG. 28 is an isometric view of the plate and the plugs 400 of FIG. 27 showing the second plugs 400 positioned between the plate 410 and the tissue 349. The position of the plugs 400 in FIG. 28 may better reduce needle hole bleeding because the plugs 400 are positioned against the corresponding needle hole. The configuration of FIG. 28 may be easier to install because you can tie down the plugs 400 first, and then optionally add on the plate 410, for example.

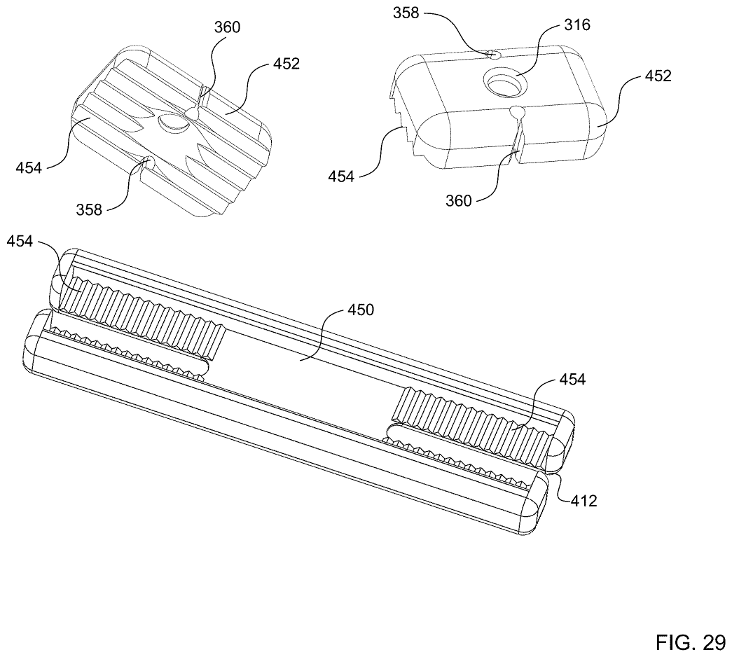

FIG. 29 is an isometric view of another plate 450 and a pair of second plugs 452 that can be used to implement the tie assembly 300 of FIG. 3. The plate 450 and the second plugs 452 have corresponding teeth 454 that mate with each other and enable linear adjustment of the second plugs 452 along the plate 450. The tie 304 being tightened in operation may secure the relative position of the second plugs 400 on the plate 450 based on the engagement, for example.

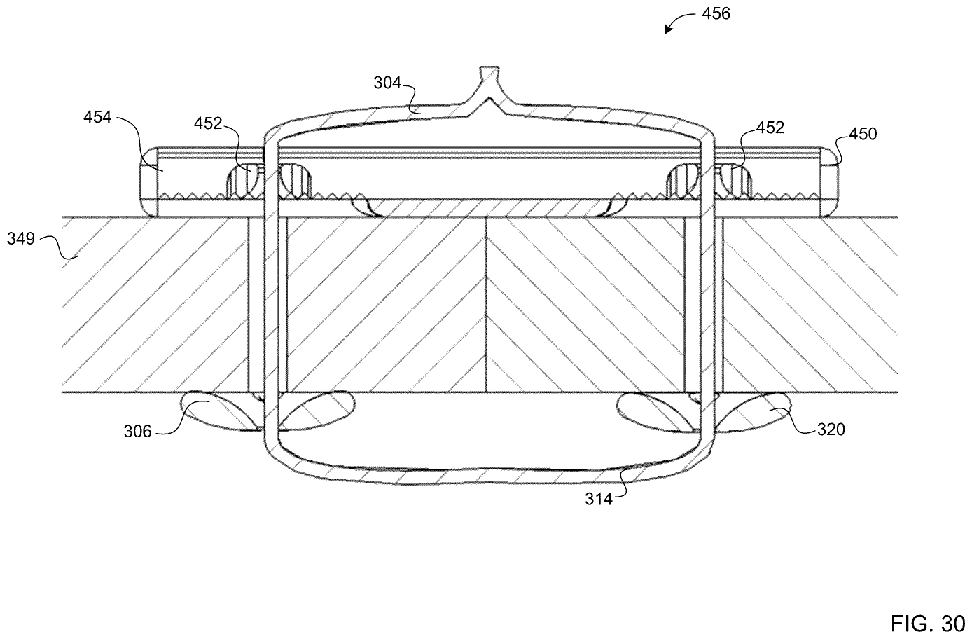

FIG. 30 shows a cross-sectional view of a tie assembly 456 including the plate 450 and the second plugs 452 of FIG. 29. The tie 304 extends through the posterior plugs 306, 320 and the anterior plugs 452 and is shown twisted together.

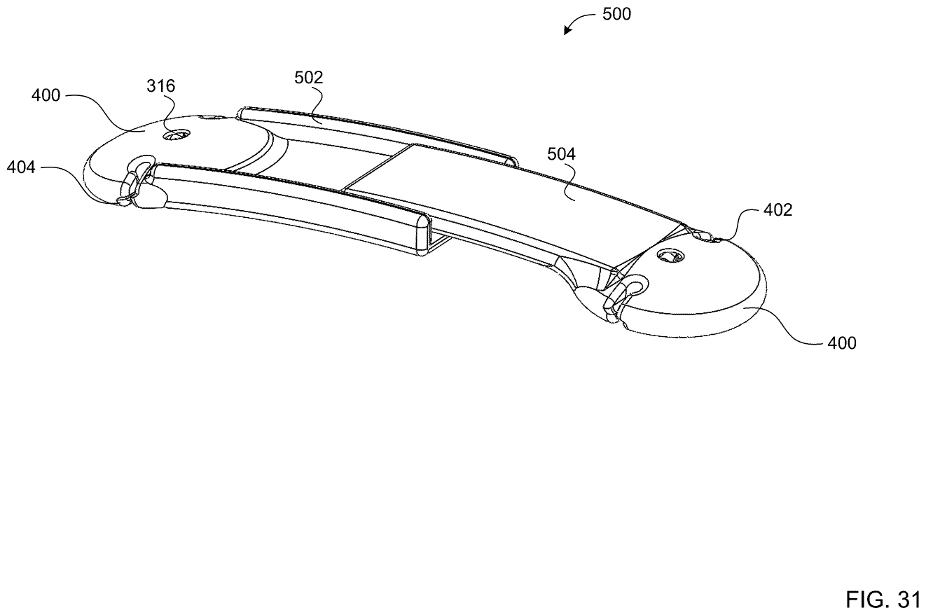

FIG. 31 is an isometric view of a second plug plate assembly 500 that can be used with the tie assembly 300 of FIG. 3. The second plug plate assembly 500 includes a pair of plates 502, 504 that each include a second plug 400. The plates 502, 504 are coupled and, in the implementation shown, the plates 502, 504 are coupled by a tongue-and-groove connection. The tongue-and-groove connection allows the plates 502, 504 to move relative to one another to accommodate different sternal widths. The tie 304 being twisted together may move the plates 502, 504 toward one another and/or together to secure the anterior plugs 400 in place.

The plates 502, 504 may be flat and/or curved to accommodate the anatomic shape of the sternum.

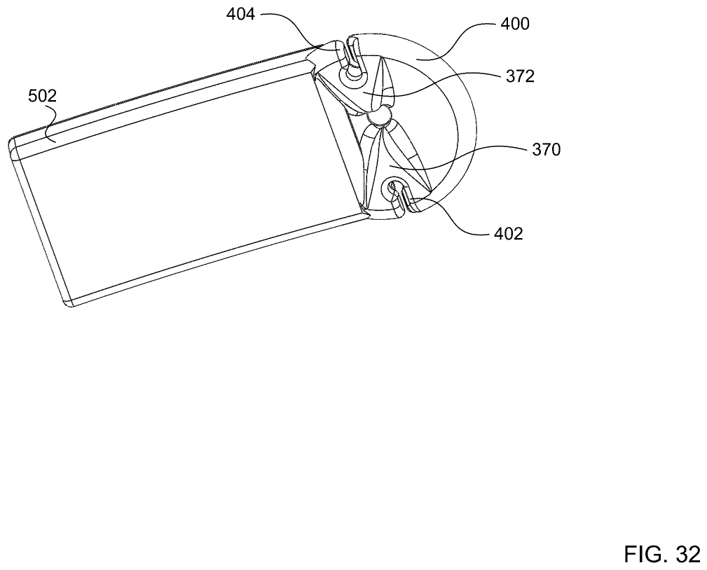

FIG. 32 is a bottom view of the plate 502 of the second plug plate assembly of FIG. 31 showing the second plug 400.

While various embodiments have been described herein, it is understood that variations can be made thereto that are still within the scope of the appended claims.

1. A tie assembly comprising:
a needle having a proximal end and a distal end;
a tie coupled to the proximal end of the needle;
a plug having an axial bore therethrough, the tie disposed within the bore; and
a tether tethering the plug to the proximal end of the needle.

2. The tie assembly of claim 1, wherein the tie comprises a wire.

3. The tie assembly of claim 1, wherein the needle, the tie, and the tether are crimped together.

4. The tie assembly of claim 1, further comprising:
a second needle having a proximal end and a distal end;
the tie extending from the proximal end of the second needle;
a second plug having an axial bore therethrough, the tie disposed within the bore; and
a second tether tethering the second plug to the proximal end of the needle.

5. (canceled)

6. The tie assembly of claim 1, wherein the plug comprises a pair of holes and wherein the tether comprises a first end, a central portion, and a second end, the first end and the second end of the tether are coupled to the proximal end of the needle and the central portion of the tether passes through the pair of holes.

7. (canceled)

8. The tie assembly of claim 1, wherein the plug has a top surface that defines an opening of the bore, a bottom surface, and a curved surface that defines the bore and curves outwardly from the opening of the bore at the top surface toward the bottom surface.

9. The tie assembly of claim 8, wherein the top surface is convex and wherein the bottom surface is convex.

10-11. (canceled)

12. The tie assembly of claim 1, wherein the needle is removable from the tie and the tether, and further comprising a second plug having an axial bore and threadable onto an exposed end of the tie upon removal of the needle.

13. The tie assembly of claim 12, wherein the second plug has a top surface that defines an opening of the bore, a bottom surface, and a curved surface that defines the bore and curves outwardly from the opening of the bore at the top surface toward the bottom surface.

14. The tie assembly of claim 12, wherein the second plug comprises a pair of grooves and wherein the tether has portions that pass through the pair of grooves.

15. The tie assembly of claim 14, wherein the second plug includes a perimeter and the grooves include tapered surfaces, an entrance, and an opening, the tapered surfaces extend inward from the perimeter of the second plug toward the entrance of the opening of the corresponding groove.

16. The tie assembly of claim 14, wherein the grooves each comprise a first portion and a second portion that are off-set from one another.

17. The tie assembly of claim 16, wherein the first portion and the second portion are radially spaced from one another.

18. The tie assembly of claim 12, wherein the second plug comprises a pair of protrusions extending from the bottom surface and positioned on opposite sides of the bore.

19. (canceled)

20. The apparatus of claim 18, wherein the protrusions define a groove between the protrusions.

21. The apparatus of claim 20, wherein the protrusions comprise outwardly tapered walls and wherein the tie interacts with the outwardly tapered walls to urge the tie to be positioned within the groove.

22-48. (canceled)

49. A tie assembly comprising:
a first needle having a proximal end and a distal end;
a tie coupled to the proximal end of the first needle;
a first posterior plug having an axial bore therethrough and holes, the tie disposed within the axial bore of the first posterior plug;
a tether passing through the holes of the first posterior plug and tethering the first posterior plug to the proximal end of the first needle;
a second needle having a proximal end and a distal end;
the tie extending from the proximal end of the second needle;
a second posterior plug having an axial bore therethrough and holes, the tie disposed within the axial bore of the second posterior plug; and
a second tether passing through the holes of the second posterior plug and tethering the second plug to the proximal end of the second needle,
anterior plugs, each anterior plug, comprising:
an axial bore;
grooves that each comprise a first portion and a second portion that are off-set from one another,
wherein the axial bore of the anterior plug is threadable onto an exposed end of the tie upon removal of the needle or the second needle, and
wherein the tether has portions that are to pass through the grooves.

50. The tie assembly of claim 49, wherein the holes of the first posterior plug comprise a pair of holes and wherein the holes of the second posterior plug comprise a pair of holes.

51. The tie assembly of claim 49, wherein each anterior plug comprises a top surface that defines an opening of the axial bore of the posterior plug and a bottom surface.

52. The tie assembly of claim 51, wherein each anterior plug comprises a curved surface that defines the axial bore of the posterior plug and curves outwardly from the opening of the bore at the top surface toward the bottom surface.
